(19) United States
(12) Patent Application Publication
(10) Pub. No.: US 2018/0010991 A1
(43) Pub. Date: Jan. 11, 2018

(54) Methods and Apparatus for Centrifuge Fluid Production and Measurement Using Resistive Cells

(71) Applicant: Weatherford Technology Holdings, LLC, Houston, TX (US)

(72) Inventor: John Anton Rohan, SR., Houston, TX (US)

(21) Appl. No.: 15/542,992

(22) PCT Filed: Jan. 15, 2016
(86) PCT No.: PCT/US16/13590

§ 371 Date: Jul. 12, 2017

Related U.S. Application Data
(60) Provisional application No. 62/104,406, filed on Jan. 16, 2015.

(51) Int. Cl. G01N 1/40; G01N 15/08; B04B 13/00; G01N 33/24; G01N 27/04

(52) U.S. Cl. CPC G01N 1/4077; G01N 33/24; G01N 27/041; B04B 13/00; G01N 15/082; G01N 15/088

(57) ABSTRACT

A system and method for centrifuge fluid production and measurement using resistive cells is provided. The method comprises separating an electrically conducting first fluid and a second fluid within a collection cell having a first and second section, wherein the collection cell has an electrically conductive outer wall and an inner wall having an insulating material disposed thereon. The method provides that the first and second fluids are separated from a solid disposed in the first section into the second section, the second fluid having a specific mass greater than the first fluid. The method further provides measuring, using one or more wires disposed in the second fluid and electrically connected to a resistance measuring unit within the second section, a resistivity change of the second fluid relative to the displacement of the first fluid, and communicating the resistivity change.

### BACKGROUND OF THE DISCLOSURE

Fluid production from rock samples during centrifugation is a difficult procedure, but it is essential in order to accurately determine rock formation properties such as the saturation, permeability, and capillary pressure profiles of the samples. However, the optical approaches currently being used to determine fluid production of rock sample limit the pressure, sample size, and physical configurations possible during centrifugation.

Optical imaging systems have been used commercially in centrifugation systems throughout the industry since the 1980's, and typically use electromagnetic energy such as X-ray to optically measure fluid displacement from a solid during centrifugation. Such imaging systems have been described in U.S. Pat. Nos. 4,671,102 and 4,567,373. However, these optical systems all have similar issues related to having limited pressure and temperature thresholds. This is mainly due to having optical site cells and poor imaging when oils or other fluids are induced or injected into the system. Also, these optical systems are typically stationary and must be synchronized in order to image objects spinning from 100 to 19,000 revolutions per minute.

One typical optical imaging system used in the art is shown in FIGS. 1A and 1B. As shown in FIG. 1A, a side view of a conventional centrifuge 10 is shown. The centrifuge 10 is shown having a motor 65 which is used to rotate a shaft 45. Also, the shaft 45 is connected to rotor 30, which is used to hold the optical sample holders 25 using pins 40 or other suitable instrument. The optical sample holders 25 typically contain the sample to be centrifuged as well as other fluids that are displaced by way of centrifugation. Moreover, the centrifuge 10 may contain a synchronization mechanism on the shaft 45 (not shown).

Further as shown in FIG. 1B, a side view of a sample holder 25 is illustrated according to the prior art. Sample holders 25 shown above are typically used for holding samples that represent solids from a downhole formation within an oil well, and typically comprise a core housing 83 with a chamber 79 for accommodating core sample 80. This measurement system can measure resistivity changes of the solid sample.

This measurement system can also optically measure the fluid produced from the solid sample into a sample holders 25. Because of the optical nature of the prior art systems, the core housing 83 is typically constructed of a material that is transparent to the type of radiation employed in the optical system, for example, aluminum may be employed for X-ray based optical measurement. Other systems such as the one disclosed in U.S. Pat. No. 6,272,906 have also disclosed using capacitive measurement systems in lieu of optical imaging systems.

It is therefore desirable to have a system and method for accurately measuring the properties of a solid without having the limitations known to systems currently known in the art. The subject matter of the present disclosure is directed to overcoming, or at least reducing the effects of, one or more of the problems set forth above.

### SUMMARY OF THE DISCLOSURE

By using resistive measurement methods instead of the optical techniques currently used in the art, many issues may be overcome. For example, by using a resistive collection cell having sidewalls that measure electrical resistivity, instead of the optical sample holders typically used in the art, the timing and synchronization issues known to the current optical systems can be solved. This is because, due to the resistivity measurement system being integrated within the collection cells, no external imaging system is necessary. Thus, there is no need to synchronize the centrifuge with an optical light source as is necessary with optical systems.

Also, removing the optical sensors of optical systems allows for using metallic cells having higher pressure thresholds when using live fluids. Further, because the measurements are no longer dependent on using optical sensors, oil or other fluids that may stick to the walls of the collection cell will not affect measurements as long as the conductive fluid is in contact with the resistive measurement system.

Moreover, by using resistive collection cells larger solid sample sizes may be used. This is because the boundary restraints are no longer necessary, as in the case of optical sample holders. Resistive collection cells may also be used at much lower speeds than optical collection cells and still maintain optimal results.

Thus, in order to overcome, or at least reduce the effects of one or more of the problems set forth above, a system and method for centrifuge fluid production and measurement using resistive cells is provided.

The method comprises separating an electrically conducting first fluid and a second fluid within a collection cell having a first and second section, wherein the collection cell has an electrically conductive outer wall and an inner wall having a non-conducting material disposed thereon. The method provides that the first and second fluids are separated from a solid disposed in the first section into the second section, the second fluid having a specific mass greater than the first fluid.

The method further provides measuring, using one or more wires disposed in the second fluid and electrically connected to a resistance measuring unit within the second section, a resistivity change of the second fluid relative to the displacement of the first fluid, and communicating the resistivity change.

The method also provides, measuring, using one or more wires connected to a first resistance measuring unit having the solid disposed within, the resistance of the solid, wherein the resistance of the solid changes as the first and second fluids are separated.

One embodiment disclosed herein is a method for measuring a property of a solid sample. The solid sample places in a first section of a collection cell. A first fluid is separated from the solid sample to a second section of the collection cell by rotating the collection cell about an axis of a centrifuge. A second fluid in the second section of the collection cell is displaced with the first fluid separated to the second section. One of the first and second fluids is electrically non-conductive, and the other of the first and second fluids is electrically conductive. A first change in electrical resistance is measured in the second section due to the displacement of the second fluid by the first fluid. The property of the solid sample is then determined with the measured first change.

To measure the first change, the first change in electrical resistance can be measured between at least two first points in the second section of the collection cell in electrical contact with one another through the second fluid in the second section. For example, portions of the second fluid, being the electrically conductive fluid, can be divided from one another in the second section with an insulated inner wall. An electric current can pass between at least one conductor in electrical contact with the second fluid inside the inner wall and an electrically resistive outer surface in electrical contact with the second fluid outside the inner wall.

In addition to or as an alternative to measure the first change, a second change in electrical resistance can be measured in the first section across the solid sample due to the separation of the second fluid from the solid sample.

In one arrangement, separating the first fluid from the solid sample in the first section to the second section of the collection cell can involve displacing the first fluid in the solid sample with a third electrically non-conductive fluid, such as air, oil, or the like, invading the solid sample. Also, separating the first fluid from the solid sample in the first section to the second section can involve at least separating a portion of the second fluid from the solid sample and from the first fluid.

In another arrangement, separating the first fluid from the solid sample to the second section of the collection cell can involve separating the first fluid as the electrically conductive fluid from the solid sample. In this case, the second fluid as the electrically non-conductive fluid is displaced in the second section and invades the solid sample in the first section with the displaced second fluid.

In yet another arrangement, separating the first fluid from the solid sample to the second section of the collection cell can involve separating the first fluid as the electrically non-conductive fluid from the solid sample. In this case, the second fluid as the electrically conductive fluid is displaced in the second section and invades the solid sample in the first section with the displaced second fluid.

According to another embodiment disclosed herein, a centrifuge is used for measuring a property of a solid sample. The centrifuge comprises a collection cell rotatable about an axis of the centrifuge. A first section of the collection cell holds the solid sample, and a second section of the collection cell is in communication with the first section. A non-conductive divider disposed in the second section divides portions of an electrically conductive fluid in the second section from one another. At least one conductor is in electrical contact with the electrically conductive fluid on one side of the non-conductive divider, and an electrically resistive contact is in electrical contact with the electrically conductive fluid on the other side of the non-conductive divider. An electrical measurement circuit connected to the at least one conductor and the electrically resistive contact measures a value of electrical resistance.

With this centrifuge, rotation of the collection cell about the axis separates either the electrically conductive fluid or an electrically non-conductive fluid from the solid sample to the second section. Because the centrifuge rotates the sample and collection cell, a communication circuit connected to the electrical measurement circuit can communicate information indicative of the value of the electrical resistance.

With this centrifuge, displacement of the electrically conductive fluid in the second section due the separation changes the values of the electrical resistance resulting from current passing between the at least one conductor and the electrically resistive contact in electrical contact with one another through the electrically conductive fluid. Further, the change in the electrical resistance is indicative of the property of the solid sample.

The foregoing summary is not intended to summarize each potential embodiment or every aspect of the present disclosure.

### BRIEF DESCRIPTION OF THE DRAWINGS

FIG. 1A illustrates a side view of an optical imaging system according to the prior art.

FIG. 1B illustrates a side view of a sample holder according to the prior art.

FIG. 2 illustrates a detailed view of a collection cell according to the present disclosure.

FIG. 3A illustrates a side view of the first section of the collection cell shown in FIG. 2 according to the present disclosure.

FIG. 3B illustrates an additional view of the first section of the collection cell that is used to support the core sample according to the present disclosure.

FIG. 4A illustrates a side view of the second section of the collection cell shown in FIG. 2 according the present disclosure.

FIGS. 4B-4C illustrates side views of alternative second sections of the collection cell according the present disclosure.

FIG. 5 illustrates an example resistance measurement communication system according to the present disclosure.

FIG. 6 illustrates a method for determining properties of a solid according to the present disclosure.

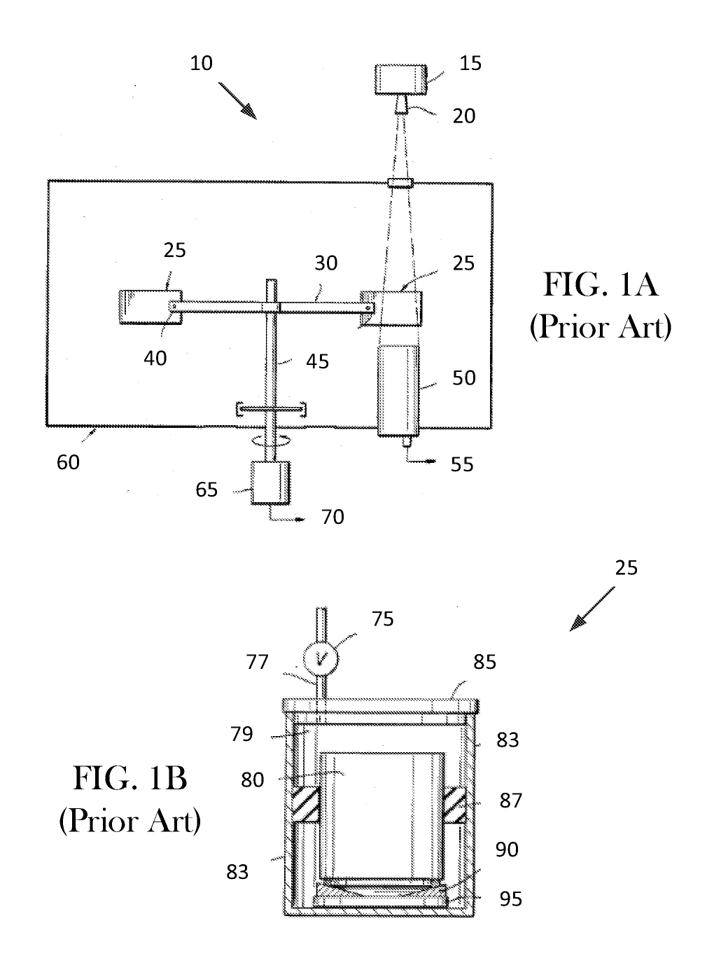

### DETAILED DESCRIPTION OF THE DISCLOSURE

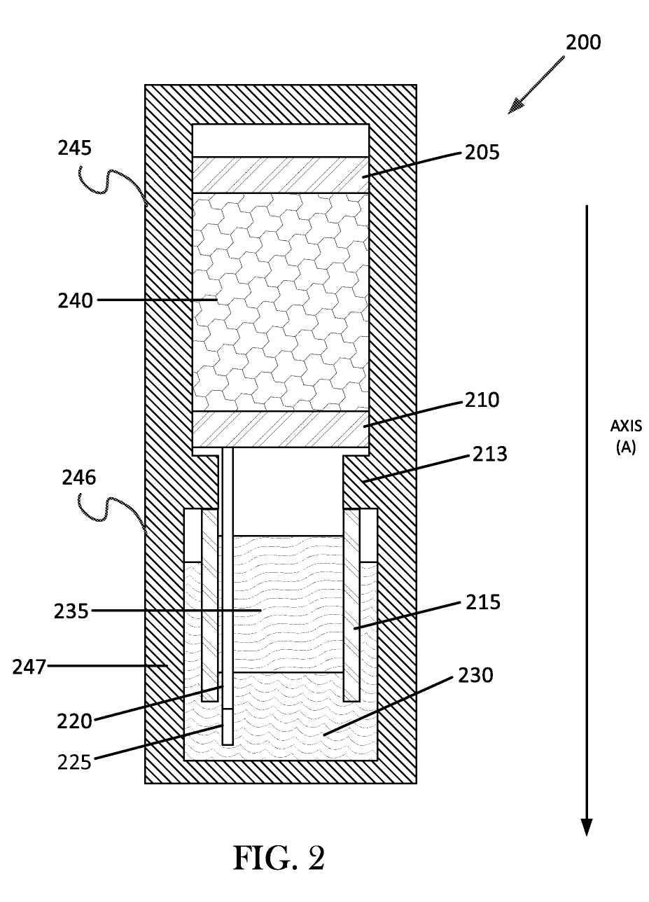

As noted previously, fluid production measurements from a sample in a centrifuge are currently done using optical measurements. These optical measurements have a number of limitations, and various corrections need to be applied to the data to deal with image blurring, meniscus changes, as well as other problems. An alternative measurement technique is disclosed herein. In one embodiment, this measurement technique uses electrical resistance to directly measure the level of an electrically conductive fluid (e.g., brine or water) in a cell as an electrically non-conductive fluid (e.g., oil) is produced from a core sample. Details of a resistive collection cell (RCC 200) are discussed below with reference to FIGS. 2-4. The system relies on the electrical conductivity of the produced fluids so considerations may need to be made for air-to-oil de-saturations.

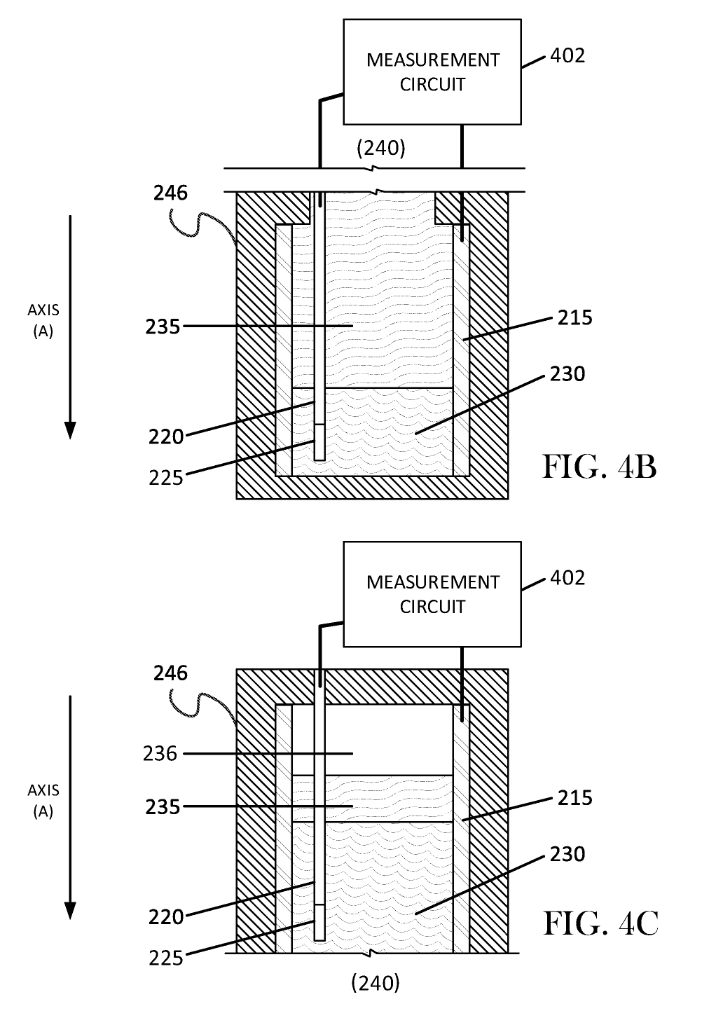

Also, after measuring the measured resistance changes from the cell 200 due to the changes in the brine or water level, the resistivity changes can be communicated wirelessly with a communication circuit because the centrifuge is rotating during the measurement process. For example, the resistivity changes can be converted directly into a frequency change and transmitted via FM transmissions or optically via a flashing LED from the centrifuge's rotor. The FM transmissions can subsequently be received and converted into fluid production measurements. Details of the above transmission techniques are discussed below with reference to FIG. 5.

Turning now to the details of the cell 200, FIG. 2 illustrates a detailed view of a cell (RCC 200) according to the present disclosure. As shown, the RCC 200 includes a first section 245 and a second section 246. The collection cell 200 is used in a centrifuge (not shown) and is rotated about a rotor with the first section 245 disposed inward toward the rotor and the second section 246 disposed radially outward from the rotor. For this configuration, the cell 200 is set up to perform production measurements in which an electrically non-conductive fluid (e.g., oil) is produced from a core sample 240 with air (or other non-conductive fluid) invading the sample 240.

As the centrifuge rotates, for example, a non-conductive oil 235 is produced from the core sample 240 in the cell 200 by being separated from the core (i.e., displaced in the core by a non-conductive gas, such as air). As the non-conductive oil 235 is produced along axis (A), a conductive fluid level from the electrically conductive fluid (e.g., brine or water) 230 in the cell 200 changes. In turn, the cell 200 registers a resistance change as a linear function of the brine or water level.

The outer wall of the RCC 200 is comprised of metal or other electrically conductive or non-conductive material, and is capable of operation using high pressures and temperatures during centrifugation. The cell 200 can be made out of Peek, fiber-glass, carbon fiber, or any other stronger material desired.

Referring to the first section 245 of the RCC 200, the solid sample 240 can be disposed within a first resistivity measurement unit (RMU) having electrodes 205 and 210. The first electrodes 205 and 210 can be used to measure the resistivity changes of the sample 240 as fluid is separated from the sample 240 into the second section 246 during centrifugation. Electrodes 205 and 210 may be, or may have disposed thereon, a conductive material that can be used for measuring the electrical resistivity of a substance, as is well known in the art.

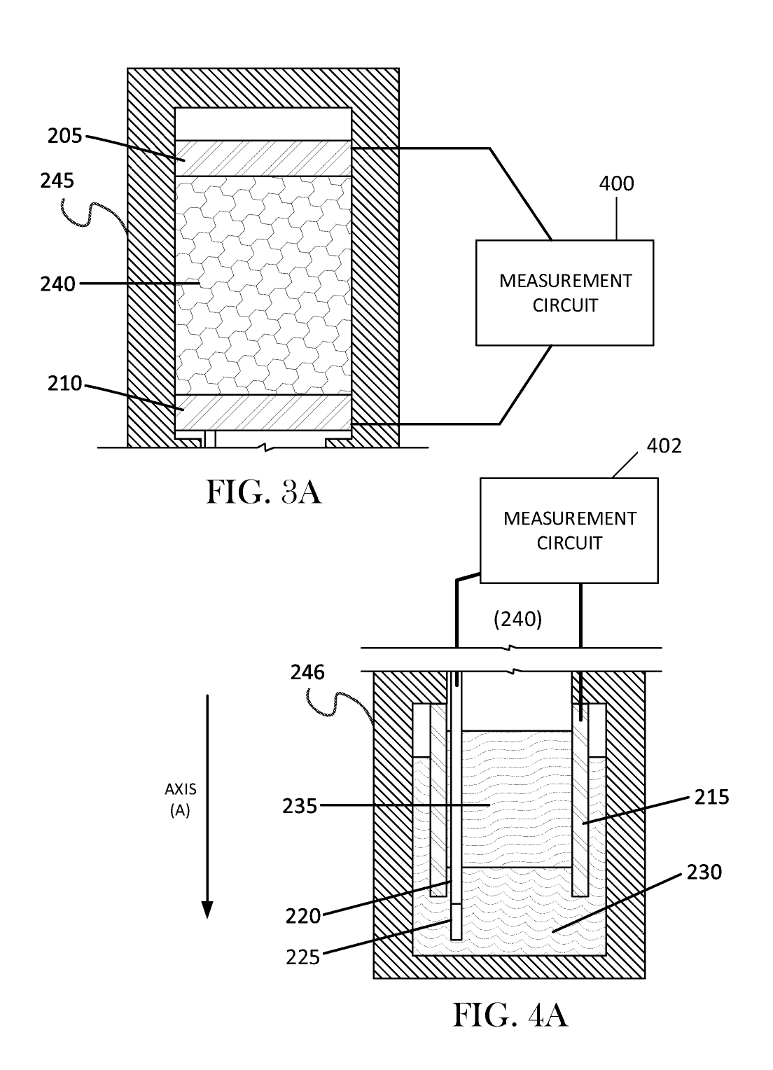

Electrodes 205 and 210 of this first RMU are further electrically isolated from the outer wall of the collection cell 200. This isolation forces electric current to flow only through the sample 240 during measurement. Also, the electrodes 205 and 210 are connected externally by wires to a measurement circuit 400 (as shown in FIG. 3A) that is used to measure the electrical resistance across the sample 240.

As described below, during centrifugation fluids within the sample 240 will be produced as a result of being separated from the sample 240 by forces along the axis (A) into the second section 246 of the collection cell 200. However, so the fluid separated from the sample 240 can be transferred into the second section 246 of the collection cell 200, one or more openings (not shown) may exist in the bottommost electrode 210 of the first RMU.

Referring to the second section 246, this second section 246 of the collection cell 200 contains a second RMU that is composed of an internal divider, wall, or cylinder 215 having a resistance material, surface, or contact insulated from contact with the outer housing 247. The resistance material of the cylinder 215 works in conjunction with at least one conductor or wire 220 having a bare contact 225 in the brine or water 230, which makes the brine 230 the other end of a resistor circuit.

The cell's second section 246 initially contains a set amount of water or brine 230. As the oil 235 produces from the sample 240 into the center annulus inside the cylinder 215, the oil 235 pushes the water 230 into the outer annulus outside the cylinder 215. As water 230 is displaced in the cell's second section 246, it effectively decreases the resistor area along the inner wall of the cylinder 215, changing the resistance measurement across the cylinder 215 and wire 220. While the oil 235 is less dense than the water 230 and would be buoyed upward, accurate data can still be produced based on any necessary corrections and calibrations.

The cylinder 215 of the second RMU extends along the major axis (A) of the collection cell 200, whereas the electrodes 205 and 210 of the first RMU are aligned perpendicular to the major axis (A).

Referring now to FIG. 3A, a side view of the first section 245 of the collection cell 200 is illustrated according to the present disclosure. This first section 245 of the collection cell 200 is adapted for use to allow resistivity measurements with or without overburden pressure during the centrifugation process. As described above, the first RMU having the electrodes 205 and 210 does not have an electrical contact with the collection cell 200. Also, because the sample 240 disposed within is electrically isolated from the collection cell 200, the electric current provided by the system will only flow through the sample 240. This will effectively isolate the sample 240 during resistivity measurements. Material used to isolate the electrodes 205 and 210 of the first RMU and/or the sample 240 may be any non-conducting isolator such as plastic, rubber, etc.

Further, the one or more wires connected to the measurement circuit 400 for the first RMU's electrodes 205 and 210 may traverse from the inside of the collection cell 200 to the outside of the collection cell 200. In this aspect, the collection cell 200 may be fitted with an end piece or cap (not shown) through which leads, wires, hoses, etc. may be passed through. Also, the one or more wires may form a circuit with the measurement circuit 400 of the first RMU's electrodes 205 and 210, and a resistance measurement communication system for measuring and communicating the resistivity across the solid sample 240 during centrifugation.

In addition to the one or more wires extending inside of the first section 245 of the collection cell 200, the collection cell 200 may have valves or ports, preferably part of the end piece or cap, for applying a preselected pressure to the solid sample 240 before or during centrifugation. Further, the collection cell 200 may have pressure or temperature measurement sensor(s) (not shown) for measuring the temperature and/or pressure of the sample 240 during centrifugation.

FIG. 3B shows an additional view of how the core sample 240 can be supported in the first section 245 of the collection cell 200 using screens 315, disks 310, and ferrules 305. As shown, the core sample 240 may be encapsulated in heat shrink Teflon tubing 322 with dual end screens 315 having a mesh structure at the top and bottom of the cell 200 next to the core sample 240.

In addition, anodized aluminum disks 310 can be used to provide the necessary tri-axial stress confinement of the sample 240, while the mesh screens 315 protect the sample 240 and serve to allow fluid only to be displaced from the sample 240 during centrifugation. As shown, the core sample 240, the screens 315, the disks 310, and the ferrules 305 may be encapsulated in the heat plastic shrink tubing 322 and can be placed in an anodized aluminum sleeve 320. A weld material can be used for overburden in the cell 200. Further, the resistance measurements of the sample 240 may be made by forming a circuit (see measurement circuit 400) using the end screens 315 on opposite sides of the sample 240. The screens 315 are isolated from each other and the outer wall of the cell 200 using the shrink tubing 322.

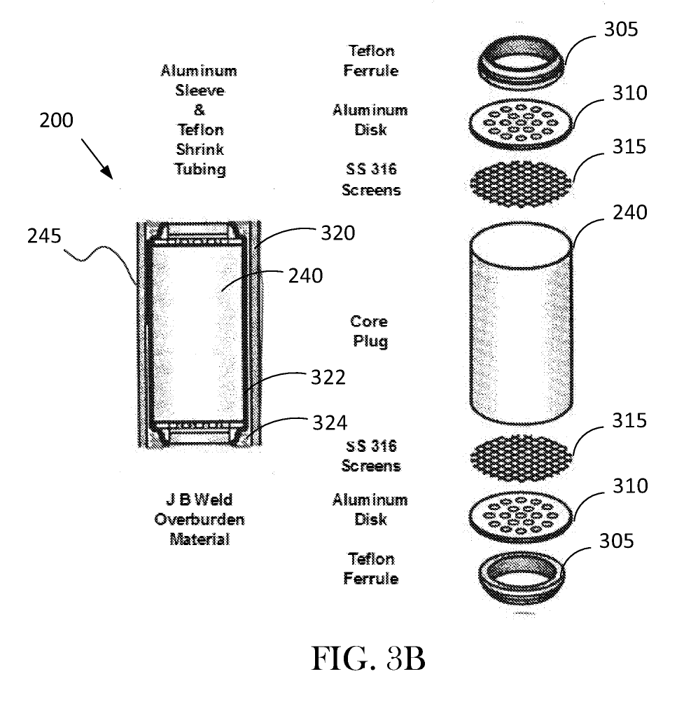

FIG. 4A illustrates a side view of the second section 246 of the collection cell 200 according the present disclosure. As shown, the second RMU's cylinder or divider 215 in the second section 246 is connected at one end to the collection cell 200. Also as shown, the RMU's cylinder or divider 215 extends from an upper portion of the second section 246, almost to the bottom of the collection cell 200, only leaving room for fluid flow under the cylinder 215.

As described below, during centrifugation the fluids (e.g., oil 235 and water 230) that are separated from the sample (240) will flow along axis (A) into the section 246, under the wall or cylinder 215, and into the annulus between wall 215 and the collection cell 200. However, to avoid an electrical connection between the collection cell 200 and the fluid, an insulating material can be disposed on the inside of the collection cell 200.

Further, one or more wires electrically connect a measurement circuit 402 to the at least one conductor or wire 220 and the electrically resistive cylinder, divider, or wall 215 of the second RMU that is extended within the second section 246. As shown, the upper portions of the one or more wires 220 extending from the first section 245 into the second section 246 are wrapped in an insulating material to prevent an electrical connection between the wires 220 and the fluids in the upper part of the second section 246. However, the bottommost end 225 of the wire 220 is bare in order to allow an electrical connection with the fluid (e.g., water or brine) at the bottommost part of the second section 246, which is the fluid having the greater specific weight in relation to the other fluids.

As a result of the above setup, the electrical connection for the measurement circuit 402 is formed between the wire 220 and the resistive cylinder 215 through the conductive fluid in the second section 246. The resistivity changes due to fluid displacement are measured between the one or more wires 220 and the resistive material along the cylinder 215. The one or more wires 220 and the cylinder 215 are connected to the resistance measurement communication system (not shown) for measuring and communicating resistivity changes of the one or more fluids. In this way, for example, portions of the electrically conductive fluid (water or brine) are divided from one another in the second section 246 with the insulated inner cylinder, wall, or divider 215. The circuit 402 can pass an electric current between wire's contact 225 in electrical contact with the fluid inside the inner cylinder 215 and the electrically resistive outer surface or contact in electrical contact with the second fluid outside the inner cylinder 215.

The second section 246 of the collection cell 200 may also have extensions 213 which may be some portion along the upper edge of the second section 246 that protrudes, and may contain and separate the components within the first section 245 from that of the second section 246. Although extensions alone have been shown, other devices may be used such as a washer or other separator that is capable of separating the first and second sections 245 and 246, while allowing fluid transfer from the first section 245 to the second section 246.

Also, along with separating the first and second sections 246, the extensions 213 may allow one or more wires 220 to traverse between the two sections. As will be discussed below, the one or more wires 220 are connected externally to a resistance measurement communication system (not shown) for receiving and communicating changes in resistivity. Further, during centrifugation, an area of the one or more wires 220 extending through to the second section 246 is protected by an insulator, while the end of the one or more wires 220 terminate having a bare end contact 225.

As discussed above, the second section 245 of cell 200 as shown in FIG. 4A is used to measure oil production from the sample (240) with invading air entering the sample (240). This technique allows for measuring a property, such as permeability or porosity, of the core sample (240) using in-situ fluids (e.g., oil) from the formation of interest. The integrity of the cell 200 can allow in-situ conditions of fluid and pressure to be applied to the core sample (240) and the fluids to mimic the conditions of live hydrocarbons in the formation. This can have particular benefits in determining the characteristics of the formation's permeability, porosity, and the like.

However, the cell 200 can further be used for other forms of production measurements, such as water production with air or oil invading, or oil production with water invading. To achieve these other production measures, changes to the electrical connections for the second measurement circuit 402 would be necessary to measure changing resistance due to displaced fluids.

For example, FIG. 4B illustrates a configuration of the cell's second section 245 used for water production from the sample (240) with air or oil invading the sample (240). In this case, a conductive fluid (e.g., water 230) is produced from the sample (240) along axis (A) and is replaced by a non-conductive fluid (e.g., oil 235 or air). As shown, as water 230 is produced from the sample (240) along the axis (A) into the second section 245 of the cell 200, the changes in the water level will correlate directly to a change in the measured resistivity as the cell 200 fills. A resistive cylinder 215 in the cell 200 measures the change in resistivity as the water 230 level changes.

As shown in a similar manner described in FIG. 4A, the cylinder 215 in the second section 246 is connected at one end to the collection cell 200. However, instead of extending almost to the bottom of the cell 200 as in FIG. 4A, the cylinder extends from the upper portion of the second section 246 to the bottom of the collection cell 200, preventing water 230 from flowing under the wall or cylinder 215. One or more wires further electrically connect a measurement circuit 402 to one or more conductive wires 220 and the resistive cylinder 215 of the second RMU within the second section 246.

As shown, the upper portions of the one or more wires 220 extending into the second section 246 are wrapped in an insulating material to prevent an electrical connection between the wires 220 and the fluids in the upper part of the second section 246. However, the bottommost end 225 of the wire 220 is bare in order to allow an electrical connection with the fluid (e.g., water or brine) at the bottommost part of the second section 246, which is the fluid having the greater specific weight in relation to the other fluids.

In addition, similar to the configuration in FIG. 4A, the electrical connection for the measurement circuit 402 is formed between the wire 220 and the resistive cylinder 215 through the conductive fluid (i.e., water 230) in the second section 246. As water is produced from the sample (240) and replaced by invading oil or air, the resistivity changes due to the fluid displacement are measured between the one or more wires 220 and the resistive material along the cylinder 215. Moreover, the one or more wires 220 and the cylinder 215 are connected to the resistance measurement communication system (not shown) for measuring and communicating resistivity changes of the one or more fluids.

As before, the second section 246 of the collection cell 200 may also have extensions (e.g., 213: FIG. 4A) which may be some portion along the upper edge of the second section 246 that protrudes, and may contain and separate the components within the first section 245 from that of the second section 246. Although extensions alone have been shown, other devices may be used such as a washer or other separator that is capable of separating the first and second sections 245 and 246, while allowing fluid transfer from the first section 245 to the second section 246.

In another example, FIG. 4C illustrates a configuration of the cell's second section 246 used for non-conductive fluid (e.g., oil 235) being produced from the core sample (240) while a conductive fluid (e.g., water 230) is invading the core sample (240) in an imbibition measurement. This configuration is the reverse of the water production configuration of FIG. 4A in that the resistance changes in the opposite direction. In this configuration, the liquid level in the second section 246 does not change so measurement of the water reduction is directly related to the amount of oil 235 produced from the core sample 240.

As shown in a similar manner described in FIG. 4A, the cylinder 215 is connected at one end to the collection cell 200 and extends from an upper portion of the second section 246 to the bottom of the collection cell 200, preventing water 230 from flowing under the wall or cylinder 215. One or more wires further electrically connect a measurement circuit 402 to one or more conductive wires 220 and the resistive cylinder 215 of the second RMU within the second section 246. However, instead of one or more wires extending from the first section 245 into the second section 246, the one or more wires may be disposed into the second section 246 of the cell 200 in another fashion. As discussed above, the collection cell 200 may be fitted with an end piece or cap (not shown) through which leads, wires, hoses, etc. may be passed through.

As shown in FIG. 4C, portions of the one or more wires 220 extending through to the second section 246 are wrapped in an insulating material to prevent an electrical connection between the wires 220 and fluids other than the water or brine. However as shown, the end 225 of the wire 220 is bare in order to allow an electrical connection with the water or brine.

Also, similar to the configuration in FIG. 4A, the electrical connection for the measurement circuit 402 is formed between the wire 220 and the resistive cylinder 215 through the conductive fluid in the second section 246. The resistivity change due to fluid displacement is measured between the one or more wires 220 and the resistive material along the cylinder 215. Also, the one or more wires 220 and the cylinder 215 are connected to the resistance measurement communication system (not shown) for measuring and communicating resistivity changes of the one or more fluids.

As before, the second section 246 of the collection cell 200 may also have extensions (213: FIG. 4A), which may be some portion along the upper edge of the second section 246 that protrudes, and may contain and separate the components within the first section 245 from that of the second section 246. However, other devices may be used such as a washer or other separator that is capable of separating the first and second sections 245 and 246, while allowing fluid transfer from the first section 245 to the second section 246.

Again, this particular imbibition measurement will produce a non-conductive fluid such as oil 235 from the core sample (240) while a conductive fluid such as water 230 enters the core sample 240 along axis (A). This measurement is the reverse of the measurements in FIGS. 4A and 4B in that as the oil 235 is produced in those measurements of FIGS. 4A-4B and as the water 230 level decreases, the measured resistance changes in the opposite direction. However, in this imbibition measurement of FIG. 4C, the fluid level in the cell 200 does not change so measurement of the water 230 reduction is directly related to the oil 235 production.

Now that the components of the collection cell 200 have been described, a system for measuring and communicating the resistivity changes is now discussed. By using methods such as broadcasting frequencies that correspond to changes in resistivity, the tool 200 can measure resistivity changes across the sample 240 and of the fluid during centrifugation.

The resistor configuration of the measurement circuit 402 for the collection cell 200 can be connected to a frequency network in such a way that the change in resistance produces a change in frequency. This signal can be FM modulated and transmitted from the centrifuge's rotor and received externally using a common FM radio or can light an LED for optical transmission. The demodulated frequency signal can then be fed into a microphone input of a computer and the signal converted into fluid levels or the LED signals converted to a voltage pulse using an optical sensor. By using a simple clock circuit driving an analog-switching chip all of the samples on the rotor can be sampled sequentially.

FIG. 5 illustrates an example resistance measurement communication system according to the present disclosure. As represented by the illustration in FIG. 5, using a simple clock circuit to drive an analog switching chip, multiple collection cells 200 (e.g., buckets 1-4) being centrifuged can be sampled sequentially. The one or more wires that are connected to the respective RMUs and the collection cells 200 are connected to a simple receiving circuit such as a 555 timer as is known in the art. By using the timer as the receiving circuit, the resistance across the solids and of the fluids within each cell (e.g., buckets 1-4) may be measured.

Further, as the resistivity of the samples 240 or the fluids in each of the buckets (1-4) change, the output frequency of the timer changes as well. The output frequency change may be implemented by a frequency generator in combination with the timing circuit. Thus, there is a correlation between the changes in the resistance of the samples 240 and/or the fluid described above, and the frequency outputs of the device. In order to communicate this frequency change, the frequency signals may be modulated and transmitted using a FM transmitter coupled to the circuit.

After the FM transmitter has transmitted the corresponding signals, the signals can be received externally and demodulated using a common FM radio. The received signals can also be interpreted to cause LEDs or other similar devices to activate for optical evaluation. The demodulated signals can further be fed into the microphone input of a computer and digital signals converted into the respective resistivity or fluid levels. Further, using optical sensors, the LED signals may be converted to changes in voltage levels.

Now that the components of the resistance measurement communication system has been described, the disclosure now refers to the method of producing fluid from a sample 240 using centrifugation, and measuring the resistivity changes of both the sample 240 and the produced fluid during centrifugation.

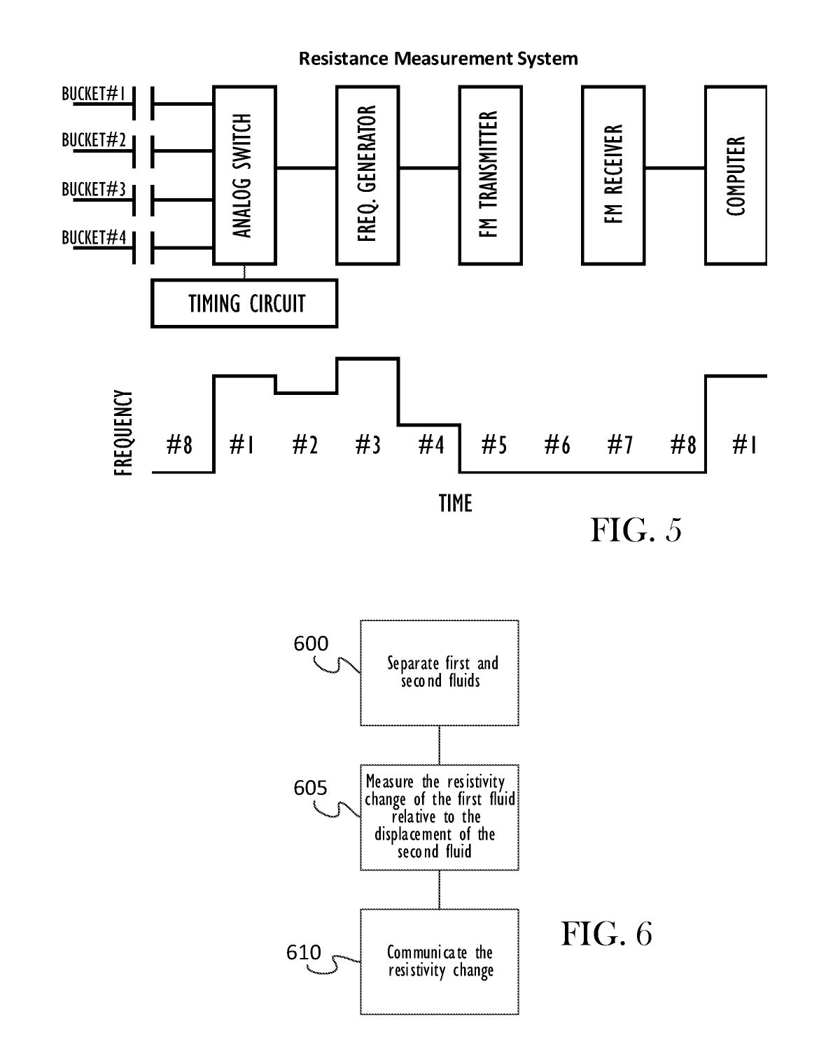

At step 600 of FIG. 6, first and second fluids are first separated from a solid sample 240 within the first section 245 of the collection cell 200 into the second section 246. As disclosed herein, this can be achieved by rotating the collection cell 200 in a centrifuge. The solid sample 240 may be a solid such as a rock or other solid, and may have a certain permeability or porosity such as rock samples found downhole in or near oil and gas wells. A first and second fluid may be displaced, wherein the first fluid is either gas or oil and the second fluid is water. As will be appreciated by those skilled in the art, the present disclosure is not limited to the above fluids as other fluids may be used for measurement.

As is typically known, water has a specific weight that is greater than that of oil; thus, as the fluids are displaced from the sample 240, the oil will be lighter and will float on the surface of the water in the collection cell 200. In addition, by the forces of centrifugation, the weight of the oil will force the water to be displaced through the inside of the second RMU (cylinder 215) to the annulus between the second RMU's cylinder 215 and the inner most portion of the second section 246 of the collection cell 200.

During the separation of the fluids, using one or more of the insulated wires 220 discussed above that extends into the second section 246 of the collection cell 200, the resistance changes due to the displacement of the fluid (i.e., water 230) can be measured at step 605. As the fluid level between the second RMU of the collection cell 200 increases, the measured resistance decreases. Also at step 605, the resistivity of the sample 240 can also be measured.

As described above, using one or more of the wires connected to the first RMU's electrodes 205 and 210 and the resistance measurement communication system, resistivity changes across the sample 240 can be measured as the fluids are separated from the sample 240 into the second section 246. The resistivity changes of either the solid sample 240 or the fluid may be measured, and at step 610 the resistivity changes may be communicated using a resistance measurement communication system as described above. Moreover, after the resistivity changes have been measured, the corresponding resistivities can be transmitted, received, and interpreted as described above with reference to the resistance measurement communication system illustrated in FIG. 5.

The foregoing description of preferred and other embodiments is not intended to limit or restrict the scope or applicability of the inventive concepts conceived of by the Applicants. As is known, the inverse of electrical resistance is electrical conductance so that any reference herein to electrical resistance can be conceptually paralleled to electrical conductance so that electrical resistance herein refers comparably to electrical conductance. Resistance is a measure of voltage across two points divided by the current passing between the points, and conductance is simply the inverse of resistance. The electrical measurements disclosed herein can be based on voltage, current, time, and other known factors. It will be appreciated with the benefit of the present disclosure that features described above in accordance with any embodiment or aspect of the disclosed subject matter can be utilized, either alone or in combination, with any other described feature, in any other embodiment or aspect of the disclosed subject matter.

In exchange for disclosing the inventive concepts contained herein, the Applicants desire all patent rights afforded by the disclosed subject matter. Therefore, it is intended that the disclosed subject matter include all modifications and alterations to the full extent that they come within the scope of the claims or the equivalents thereof.

1. A method for measuring a property of a solid sample, the method comprising:
placing the solid sample in a first section of a collection cell;
separating a first fluid from the solid sample to a second section of the collection cell by rotating the collection cell about an axis of a centrifuge;
displacing a second fluid in the second section of the collection cell with the first fluid separated to the second section, one of the first and second fluids being electrically non-conductive, the other of the first and second fluids being electrically conductive;
measuring a first change in electrical resistance in the second section due to the displacement of the second fluid by the first fluid; and
determining, with the measured first change, the property of the solid sample.

2. The method of claim 1, wherein measuring the first change comprises measuring the first change in electrical resistance between at least two first points in the second section of the collection cell in electrical contact with one another through the second fluid in the second section.

3. The method of claim 2, wherein measuring the first change in electrical resistance between the at least two first points comprises:
dividing portions of the second fluid, being the electrically conductive fluid, from one another in the second section with an insulated wall; and
passing an electric current between at least one conductor in electrical contact with the second fluid and an electrically resistive contact in electrical contact with the second fluid.

4. The method of claim 1, further comprising measuring a second change in electrical resistance in the first section across the solid sample due to the separation of the second fluid from the solid sample.

5. The method of claim 1, wherein separating the first fluid from the solid sample in the first section to the second section of the collection cell comprises displacing the first fluid in the solid sample with a third electrically non-conductive fluid invading the solid sample.

6. The method of claim 1, wherein separating the first fluid from the solid sample in the first section to the second section comprises at least separating a portion of the second fluid from the solid sample and from the first fluid.

7. The method of claim 1, wherein separating the first fluid from the solid sample to the second section of the collection cell comprises separating the first fluid as the electrically conductive fluid from the solid sample; and wherein displacing the second fluid in the second section of the collection cell with the first fluid separated to the second section comprises displacing the second fluid as the electrically non-conductive fluid and invading the solid sample in the first section with the displaced second fluid.

8. The method of claim 1, wherein separating the first fluid from the solid sample to the second section of the collection cell comprises separating the first fluid as the electrically non-conductive fluid from the solid sample; and wherein displacing the second fluid in the second section of the collection cell with the first fluid separated to the second section comprises displacing the second fluid as the electrically conductive fluid and invading the solid sample in the first section with the displaced second fluid.

9. A centrifuge for measuring a property of a solid sample, the centrifuge comprising:
a collection cell rotatable about an axis of the centrifuge;
a first section of the collection cell holding the solid sample;
a second section of the collection cell in communication with the first section;
a non-conductive divider disposed in the second section and dividing portions of an electrically conductive fluid in the second section from one other;
at least one conductor in electrical contact with the electrically conductive fluid;
an electrically resistive contact in electrical contact with the electrically conductive fluid; and
an electrical measurement circuit connected to the at least one conductor and the electrically resistive contact and measuring a value of electrical resistance.

10. The centrifuge of claim 9, wherein rotation of the collection cell about the axis separates either the electrically conductive fluid or an electrically non-conductive fluid from the solid sample to the second section.

11. The centrifuge of claim 10, wherein displacement of the electrically conductive fluid in the second section due the separation changes the values of the electrical resistance resulting from current passing between the at least one conductor and the electrically resistive contact in electrical contact with one another through the electrically conductive fluid.

12. The centrifuge of claim 11, wherein the change in the electrical resistance is indicative of the property of the solid sample.

13. The centrifuge of claim 9, further comprising a communication circuit connected to the electrical measurement circuit and communicating information indicative of the value of the electrical resistance.
